(19) United States
(12) Patent Application Publication
(10) Pub. No.: US 2026/0183262 A1
(43) Pub. Date: Jul. 2, 2026

(54) RNA-BINDING MOTIF PROTEIN 39 (RBM39) DEGRADERS FOR TREATMENT OF CANCERS

(71) Applicant: RECURSION PHARMACEUTICALS, INC., Salt Lake City, UT (US)

(72) Inventor: Ashish Ashok Bhandari, Cincinnati, OH (US)

(21) Appl. No.: 19/132,000

(22) PCT Filed: Dec. 8, 2023
(86) PCT No.: PCT/US2023/083032

§ 371 Date: May 22, 2025

Related U.S. Application Data
(60) Provisional application No. 63/431,055, filed on Dec. 8, 2022.

(51) Int. Cl. A61K 31/404; A61K 31/427; A61K 31/454; A61K 31/502; A61P 35/00

(52) U.S. Cl. CPC A61K 31/404; A61K 31/427; A61K 31/454; A61K 31/502; A61P 35/00

(57) ABSTRACT

A method for treating a homologous recombination (HR)-deficient cancer, a homologous recombination (HR)-proficient cancer or a cancer that is resistant to DNA repair and DNA damage response (DDR) inhibitor therapy, such as Poly (ADP-Ribose) Polymerase (PARP) inhibitor therapy, is provided, the method comprising administering to a subject an RNA-binding motif protein 39 (RBM39) degrader, such as an aryl sulfonamide. A DDR inhibitor, such as a PARP inhibitor, may also be administered. A composition comprising a RBM39 degrader, such as an aryl sulfonamide, e.g., E7820 or Compound A, and a DDR inhibitor, such as a PARP inhibitor, is also provided.

### FIELD

The present disclosure provides a method of treating (i) homologous recombination (HR)-deficient cancers, (ii) homologous recombination (HR)-proficient cancers and (iii) cancers that are resistant to DNA repair and DNA damage response (DDR) inhibitor therapy, using an RNA-binding motif protein 39 (RBM39) degrader. The RBM39 degrader may be combined with a DDR inhibitor, such as a Poly (ADP-Ribose) Polymerase (PARP) inhibitor, and the disclosure further relates to a composition comprising a RBM39 degrader and a DDR inhibitor, such as a PARP inhibitor.

### BACKGROUND

The DNA repair and DNA damage response (DDR) is frequently disrupted in a wide variety of cancers and is considered a hallmark of cancer. Cancer cells that harbor defects in the DDR pathway accumulate genomic instability that contributes to their aggressive behavior. Nevertheless, tumors can compensate for these defects by relying on alternative pathways for repair, leading to their survival. This provides an opportunity for targeted therapeutics that can be designed to selectively inhibit these alternative repair pathways and induce synthetic lethality (Brown, J S, et al. Cancer Discov., 2017, 7(1): 20-37).

Several large-scale genomic datasets highlight that numerous cancers such as ovarian cancers, breast cancers, prostate cancers, pancreatic cancers, non-small cell lung cancers, and small-cell lung cancers harbor molecular alterations within the DDR repair network (Knijnenburg T A, et al. Cell Rep., 2018, 23(1): 239-254. Sen T. et al. Transl Lung Cancer Res., 2018, 7(1):50-68). While patients harboring genetic abnormalities in the DDR pathway such as homologous recombination (HR) can be treated with DDR inhibitors, such as Poly (ADP-Ribose) Polymerase (PARP) inhibitors, many cancers will ultimately develop resistance (Brown, J S, et al. Cancer Discov., 2017, 7(1): 20-37). Additionally, a large proportion of patients do not derive significant benefit from DDR/PARP inhibition as they lack DDR genetic deficiencies. These patients are often referred to as homologous recombination deficiency negative (HRD-negative), homologous recombination proficient (HRP), or homologous recombination repair negative (HRR negative) and have poor prognosis with limited treatment options. Accordingly, there is a need for developing therapeutics that can regulate DNA repair mechanisms for the treatment of HRD negative, HRP, or HRR negative cancers, and DDR/PARP inhibitor resistant cancers.

Cyclin-dependent kinases (CDKs) are a group of serine/threonine protein kinases that play critical roles in various biological processes by regulating cell cycle and gene transcription. Recent reports have identified cyclin-dependent kinase 12 (CDK12) as a transcriptionally associated CDK that forms a complex with cyclin K (CCNK) and phosphorylates RNA polymerase II (RNAP2) to initiate the transcriptional elongation of several genes related to DNA damage response, cell cycle control, RNA splicing, and the maintenance of genomic stability (Dubbury, S., et al. Nature., 2018, 564, 141-145. Liang S, et al. Cells., 2020 9(6):1483).

Several reports suggest that genetic or pharmacologic depletion of CDK12 can reduce the expression of several genes involved in the homologous recombination repair pathway such as BRCA1 and BRCA2, inducing a BRCAness like phenotype that may lead to a synthetic lethality phenotype when combined with DDR inhibitors such as PARP inhibitors. Preclinical studies have demonstrated this synergistic combination in cancer cell line derived and patient derived mouse models. Additionally, genome-wide studies suggest that CDK 12 deficiency may predict sensitivity to DDR/PARP inhibitors in the clinic. Thus, CDK12 has received considerable interest as a therapeutic target and tumor biomarker for patients who develop resistance to DDR/PARP inhibitors or in combination with DDR/PARP inhibitors for HRD negative tumors (Bajrami I., et al. Cancer Res., 2014, 74(1):287-97. Johnson S F, et al. Cell Rep., 2016 Nov. 22; 17(9):2367-2381).

CDK12 has also been reported to share a largely conserved kinase domain with CDK13, with its biological role not fully understood. Genetic depletion studies suggest that while not entirely redundant, the dual inhibition of both CDK12 and CDK13 is responsible for the pronounced global transcriptional changes induced as a result of these kinases increasing RNA polymerase II processivity versus either alone. Given that CDK12 inhibition predominantly affects DNA damage response gene expression, selective targeting of either CDK12 or CDK13 in combination with PARP inhibitors may reduce unwarranted side effects associated with global RNA polymerase II disruption (Fan, Z., et al. Sci Adv., 2020, 6(18):eaaz5041. Krajewska, M., et al. Nat Commu., 2019, 10:1757). Therefore, developing pharmacologically selective CDK12 inhibitors is a major challenge as CDK12 and CDK13 share highly similar sequences.

Aryl sulfonamides function as molecular glue degraders of RNA-binding motif protein 39 (RBM39) by forming a ternary complex with RBM39 and the E3 ubiquitin ligase receptor DDB1 and CUL4 associated factor 15 (DCAF15), with no detectable affinity for either species alone. These molecular glues promote the interaction of the RBM39 splicing factor and the CUL4-DCAF15 E3 ubiquitin ligase, leading to polyubiquitination and proteasomal degradation of RBM39. In human cancer cell lines treated with aryl sulfonamides, the degradation of RBM39 led to significant anti-proliferative effects. Additionally, using CRISPR-Cas9 to silence DCAF15 in cancer cells resisted RBM39 degradation by aryl sulfonamides, highlighting RBM39 degradation as the primary mechanism of anticancer effects seen with these compounds (Han, et al., Science., 2017, 356(6336). Du, et al. Structure., 2019, 1625-1633). Furthermore, genetic knockout experiments of RBM39 deficient human cancer cells injected into mice slowed the growth of leukemia progression and improved overall survival (Wang, et al. Cancer Cell., 2019, 35(3): 369-384).

Aryl sulfonamides have previously been shown to exhibit an acceptable safety profile in clinical trials, with some anti-tumor efficacy seen across various cancers. Therefore, RBM39 degraders have the potential to effectively treat certain types of human cancers warranting further exploration (Wang, et al. Cancer Cell., 2019, 35(3): 369-384). However, overall response rates remain low, potentially due to a lack of understanding around the mechanism of action and potential biomarkers of response.

### SUMMARY

Aspects of the disclosure relate to, in part, the surprising discovery that a similar functional relationship exists specifically for the transcriptional regulation of homologous recombination (HR) repair mechanisms and cell-cycle checkpoint control for (i) RNA-binding motif protein 39 (RBM39) and (ii) cyclin-dependent kinase 12 (CDK12). RBM39 degraders may therefore be used to treat cancers where CDK12 is a suitable therapeutic target, for example, cancers where patients develop resistance to DDR/PARP inhibitors or cancers with HRD negative/HR proficient tumors. Advantageously, the inventors have shown that RBM39 degradation can phenocopy CDK12 inhibition with minimal impact to other cyclin dependent kinases and thus reduce unwarranted side effects associated with global RNA polymerase II disruption.

Accordingly, a first aspect of the disclosure provides a method for treating a homologous recombination (HR)-deficient cancer in a subject in need thereof, the method comprising administering to the subject an RNA-binding motif protein 39 (RBM39) degrader in an amount effective to treat the HR-deficient cancer.

The present disclosure therefore provides an RBM39 degrader for use in the treatment of a homologous recombination (HR)-deficient cancer in a subject in need thereof.

The method may further comprise administering a DNA repair and DNA damage response (DDR) inhibitor to the subject in an amount effective to treat the cancer.

A second aspect of the disclosure provides a method for treating a homologous recombination (HR)-proficient cancer in a subject in need thereof, the method comprising administering to the subject an RNA-binding motif protein 39 (RBM39) degrader in an amount effective to treat the HR-proficient cancer.

The present disclosure therefore provides an RBM39 degrader for use in the treatment of a homologous recombination (HR)-proficient cancer in a subject in need thereof.

The method may further comprise administering a DNA repair and DNA damage response (DDR) inhibitor to the subject in an amount effective to treat the cancer.

According to a third aspect of the disclosure, there is provided a method for treating a cancer that is resistant to DNA repair and DNA damage response (DDR) inhibitor therapy, such as poly (ADP-ribose) polymerase (PARP) inhibitor therapy, in a subject in need thereof, the method comprising administering to the subject an RNA-binding motif protein 39 (RBM39) degrader in an amount effective to treat the cancer that is resistant to the DDR inhibitor therapy.

The present disclosure therefore also provides an RBM39 degrader for use in the treatment of a cancer that is resistant to DDR inhibitor therapy, such as PARP inhibitor therapy, in a subject in need thereof.

The method may further comprise administering a DNA repair and DNA damage response (DDR) inhibitor to the subject in an amount effective to treat the cancer.

According to a fourth aspect of the present disclosure, there is provided a composition comprising an RNA-binding motif protein 39 (RBM39) degrader and a DNA repair and DNA damage response (DDR) inhibitor, for example a poly (ADP-ribose) polymerase (PARP) inhibitor.

According to a fifth aspect of the disclosure, there is provided a method of predicting a cancer subject's response to treatment with an RNA-binding motif protein 39 (RBM39) degrader, the method comprising determining the subject's response to treatment with a DNA repair and DNA damage response (DDR) inhibitor therapy, such as poly (ADP-ribose) polymerase (PARP) inhibitor therapy, wherein a subject that is resistant to DDR inhibitor therapy may benefit from treatment with a RBM39 degrader. The method may comprise administering an RBM39 degrader to the subject in an amount effective to treat the cancer wherein the subject is identified as being resistant to DDR inhibitor therapy. The method may further comprise administering a DNA repair and DNA damage response (DDR) inhibitor to the subject in an amount effective to treat the cancer.

According to a sixth aspect of the disclosure, there is provided a method of predicting a cancer subject's response to treatment with an RNA-binding motif protein 39 (RBM39) degrader, the method comprising determining the subject's Homologous recombination deficiency (HRD) status. The method may comprise administering an RBM39 degrader to the subject. The method may further comprise administering a DNA repair and DNA damage response (DDR) inhibitor to the subject in an amount effective to treat the cancer. In particular, the method may comprise administering an RBM39 degrader to the subject in an amount effective to treat the cancer wherein the subject is identified as being HR-deficient. The RBM39 degrader may be administered as a single agent or in combination with a DDR inhibitor wherein the subject is identified as being HR-deficient. The method may comprise administering both an RBM39 degrader and a DDR inhibitor to the subject wherein the subject is identified as being HR-proficient.

### BRIEF DESCRIPTION OF THE FIGURES

The disclosure will be more clearly understood from the following description of some embodiments thereof, given by way of example only, with reference to the following figures in which:

FIG. 1A illustrates tumor volume results and FIG. 1B illustrates tumor growth inhibition (TGI) for treatment with RBM39 degrader E7820 single agent or in combination with olaparib in the HRD negative OVCAR3 CDX model;

FIG. 2A-H illustrates pharmacodynamic markers from tumor samples harvested from the in vivo CDX study of Example 1, FIG. 2A for BRCA1; FIG. 2B for MCL1; FIG. 2C for pHH3; FIG. 2D for RBM39; FIG. 2E for CDK12; FIG. 2F CDK7; FIG. 2G for CDK9; and FIG. 2H for CDK1/2/3/5;

FIG. 3A illustrates tumor volume and FIG. 3B illustrates tumor growth inhibition (TGI) results for treatment with E7820 single agent or in combination with olaparib in the BRCA-proficient and PARP resistant OV0273 PDX model;

FIG. 4A illustrates individual spider plots of mice treated with E7820 single agent and FIG. 4B shows individual spider plots of mice treated with the combination of E7820+olaparib in the BRCA 1/2 proficient and PARP resistant OV0273 PDX model;

FIG. 5 illustrates overall survival results for treatment with E7820 single agent or in combination with olaparib in the BRCA 1/2 proficient PARP resistant OV0273 PDX model;

FIG. 6 illustrates the number of complete regressions observed with E7820 single agent and E7820 in combination with olaparib in the BRCA 1/2 proficient PARP resistant OV0273 PDX model;

FIG. 7A illustrates the tumor volume and FIG. 7B illustrates the tumor growth index (TGI) results for treatment with niraparib, olaparib, E7820 in combination with niraparib, and E7820 in combination with olaparib in the BRCA-proficient OV90 CDX model;

FIGS. 8A and B show viability curves for the HRD negative human ovarian cancer cell line OVCAR3, treated with CDK12/13 inhibitors, THZ531 and SR-4835 (FIG. 8A), or RBM39 degraders, E7820 and indisulam (FIG. 8B) for 72 hours;

FIG. 9 shows CDK12 kinase activity curves in the human epithelial kidney cell line HEK293 treated with CDK12/13 inhibitor THZ531, CDK12 degrader CR8, RBM39 degraders, E7820 and indisulam, and pan-CDK inhibitor AT7519 for 1 hour; and

FIG. 10A and FIG. 10B show gene expression changes in BRCA1 and ATR (FIG. 10A) as well as additional DNA repair and cell cycle genes (FIG. 10B) in the HRD negative human ovarian cancer cell line OVCAR3, treated with E7820 for 6 hours (FIG. 10A) and 24 hours (FIG. 10B).

FIG. 11 illustrates tumor volume results (FIG. 11A), tumor growth inhibition results (FIG. 11B) and target engagement marker from tumors samples (FIG. 11C) for treatment with Compound A single agent or in combination with niraparib in the BRCA 1/2 proficient and PARP resistant OV0273 PDX mouse model.

### DETAILED DESCRIPTION

As disclosed herein, numerous small molecules have been investigated for HRD deficient and PARP inhibitor resistant cancers and it was confirmed that E7820 is efficacious alone or in combination with PARP inhibitors in preclinical mouse cancer models. To select E7820 as a potential therapeutic for this disease, a high content phenotypic whole genome arrayed CRISPR/Cas9 screen was developed in HUVEC cells to identify cellular and structural changes associated between the genetic knockout of human genes. This high dimensional biological data set of single gene knockout phenotypes was then compared to the cellular and structural changes arising from the treatment of over 200,000 small molecules in intron-controlled wild-type HUVEC cells. Using machine vision and automated analysis software, hundreds of cellular parameters associated with CDK12 knockout were quantified to elucidate therapeutic compounds that may phenocopy CDK12 loss, identifying a number of aryl sulfonamides (indisulam, tasisulam, CQS, and E7820). Utilizing the same process, it was further confirmed that all of these aryl sulfonamides also phenocopy RNA-binding motif protein 39 (RBM39) CRISPR/Cas9 knockout, validating the recently discovered mechanism that these compounds function as molecular glue degraders of RBM39 by forming a ternary complex with RBM39 and the E3 ubiquitin ligase receptor DDB1 and CUL4 associated factor 15 (DCAF15). RBM39 degraders, such as these aryl sulfonamides, may therefore be used to treat cancers where CDK12 is a suitable therapeutic target, for example, cancers where patients develop resistance to PARP inhibitors or cancers with HRD negative/HR-proficient tumors. Similar to depletion of CDK12, RBM39 degraders may be used to induce a synthetic lethality phenotype when combined with DDR inhibitors such as PARP inhibitors, thus enabling treatment of patients who would not derive benefit from treatment with the DDR inhibitor in the absence of an RBM39 degrader.

### Homologous Recombination Deficiency (HRD) Status

Homologous recombination deficiency (HRD) is a tumor characteristic that is defined by the inability to accurately repair double-strand breaks (DSBs) in DNA via homologous recombination (HR). HRD status can be assessed via the assessment of genomic instability. In particular, HRD status may be scored by a system that measures genomic defects reflective of HR deficiency, including those that quantify loss of heterozygosity (LOH), telomeric allelic imbalance, and large-scale state transitions. Deleterious mutations of certain genes (e.g., breast cancer susceptibility genes 1 or 2 (BRCA1 and/or BRCA2), genes related to the Fanconi Anemia repair pathway, ATM, TP53, genes related to the base excision repair pathway, genes related to the non-homologous end joining pathway, genes related to the alternative-end joining pathway) are correlated with HR-deficiency in some cancers. HRD status can therefore also be assessed based on the presence or absence of one or more deleterious mutations in one or more genes correlated with HR-deficiency, such as BRCA1 and/or BRCA2 genes, genes related to the Fanconi Anemia repair pathway, ATM, TP53, genes related to the base excision repair pathway, genes related to the non-homologous end joining pathway and genes related to the alternative-end joining pathway. In particular, HRD status can be assessed based on the presence or absence of individual deleterious mutations in BRCA1 and/or BRCA2 genes.

For example, for ovarian cancer, HRD status may be determined by two biomarkers: (i) germline mutations in BRCA1 and/or BRCA2 gene resulting in deleterious or benign mutations, or (ii) genomic instability level as assessed by loss of heterozygosity, telomeric allelic imbalance, and large-scale state transitions wherein the sum of these three independent measures quantifies a positive or negative status result. For prostate cancer, HRD status may be determined by germline or somatic mutations in the HRR pathway: BRCA1, BRCA2, ATM, BARD1, BRIP1, CDK12, CHEK1, CHEK2, FANCL, PALB2, RAD51B, RAD51C, RAD51D and/or RAD54L. For breast cancer and pancreatic cancer, HRD status may be determined by deleterious germline BRCA1/2 mutations wherein all patients that are BRCA1/2 positive, are also HRD positive. However, patients can be BRCA 1/2 negative, but still be HRD positive, as is the case for ovarian cancer when referring to HRD status using the second definition above. Genetic tests are available for use by clinicians to determine HRD status for clinical application of PARP inhibitors, for example, Myraid, Foundation Medicine, Caris and Tempus. For Myraid CDx test, HRD positive is defined as either a tBRCA mutation and/or an HRD score≥42 by Myriad myChoice® CDx as these were the scores seen in 95th percentile of HRD scores in BRCA 1/2 deficient patients. HRD negative is defined as either non-tBRCA-mutated and/or HRD score <42 by Myriad myChoice® CDx. HRD unknown is defined in accordance with a test that fails, is inconclusive, or missing (https://www.accessdata.fda.gov/cdrh_docs/pdf19/P190014B.pdf). For Foundation Medicine CDx test, HRD positive is defined as either a tBRCA mutation and/or an LOH score >16% (https://www.accessdata.fda.gov/cdrh_docs/pdf16/p160018S001c.pdf).

The terms HRD positive and HR-deficient are used interchangeably herein to refer to cancers/tumors having one or more markers of genomic instability, such as one or more deleterious mutations in genes correlated with HR-deficiency (e.g. BRCA1/2), and positive genomic instability (GIS). The terms HRD negative, HR-proficient and homologous recombination repair negative (HRR negative) are used interchangeably herein to refer to cancers/tumors that lack DDR genetic deficiencies, that is, cancers/tumors lacking genomic instability, such as deleterious mutations in genes correlated with HR-deficiency (e.g. BRCA1/2). The term “BRCA 1/2 proficient” as used herein refers to a cancer where the subject has functioning BRCA 1 and 2 proteins, which induces genomic stability. Genes correlated with HR-deficiency may be selected from the group consisting of BRCA genes, genes related to the Fanconi Anemia repair pathway, ATM, TP53, genes related to the base excision repair pathway, genes related to the non-homologous end joining pathway and genes related to the alternative-end joining pathway. The one or more mutations are mutations in cells and/or tumors of the cancer. The one or more mutations may be somatic mutations and/or germline mutations. The one or more mutations may result in a loss of function of the gene in question.

Accordingly, a method as disclosed herein may include a step of determining the subject's Homologous recombination deficiency (HRD) status. For example, the method may include a step of quantifying loss of heterozygosity (LOH), telomeric allelic imbalance, and/or large-scale state transitions. Additionally or alternatively, the method may include a step of determining the presence or absence of one or more deleterious mutations in one or more genes correlated with HR-deficiency, such as breast cancer susceptibility genes 1 or 2 (BRCA1 or BRCA2), genes related to the Fanconi Anemia repair pathway, ATM, TP53, genes related to the base excision repair pathway, genes related to the non-homologous end joining pathway and genes related to the alternative-end joining pathway. In particular, the method may include a step of determining the presence or absence of one or more deleterious mutations in breast cancer susceptibility genes 1 and/or 2 (BRCA1 and/or BRCA2).

### DNA Repair and DNA Damage Response (DDR) Inhibitor

A DNA repair and DNA damage response (DDR) inhibitor, such as a Poly (ADP-Ribose) Polymerase (PARP) inhibitor, may also be administered to the subject. Accordingly, the disclosed methods may include a step of administering a DDR inhibitor, such as a PARP inhibitor, to the subject.

The term “DDR inhibitor” as used herein refers to any compound that inhibits, blocks or reduces DNA repair and DNA damage response. In cancer treatment, blocking DNA repair and DNA damage response may help stop cancer cells from repairing their damaged DNA, causing them to die. The DDR inhibitor may be a Poly (ADP-Ribose) Polymerase (PARP) inhibitor. The DDR inhibitor may be an ATR inhibitor. The DDR inhibitor may be a CHK1 inhibitor. The DDR inhibitor may be selected from the group consisting of inhibitors of WEE, RAD51, ATR, PolTheta, BLM, WRN, PARG, USP1 and DNAPKc.

The term “DDR inhibitor” as used herein is understood to encompass pharmaceutically acceptable salts thereof. The term “DDR inhibitor” as used herein may refer to one DDR inhibitor or a combination of two or more DDR inhibitors.

RBM39 degraders may induce a synthetic lethality phenotype when combined with the DDR inhibitor to enable treatment of patients who would not derive benefit from treatment with the DDR inhibitor in the absence of the RBM39 degrader.

### Poly (ADP-Ribose) Polymerase (PARP) Inhibitor

Poly (ADP-Ribose) Polymerase (PARP) helps repair DNA when damaged. The term “PARP inhibitor” as used herein refers to any compound that inhibits or blocks PARP, that is, any compound that downregulates, reduces or ceases PARP expression, activity and/or function. In cancer treatment, blocking PARP may help stop cancer cells from repairing their damaged DNA, causing them to die.

The PARP inhibitor may be selected from the group consisting of olaparib, niraparib, talazoparib and rucaparib. The PARP inhibitor may be olaparib. The PARP inhibitor may be niraparib.

The term “PARP inhibitor” as used herein is understood to encompass pharmaceutically acceptable salts thereof. The term “PARP inhibitor” as used herein may refer to one PARP inhibitor or a combination of two or more PARP inhibitors.

### Cancer

The cancer may be selected from the group consisting of ovarian cancer, breast cancer, prostate cancer, gastric cancer, pancreatic cancer, KRAS mutated cancers, acute myeloid leukemia, colon cancer, neuroblastoma, hematopoietic cancers, lymphoid cancers, non-small cell lung cancer and small-cell lung cancer. The cancer may be ovarian cancer.

The cancer may be a HR-proficient cancer. The HR-proficient cancer may be selected from the group consisting of ovarian cancer, breast cancer, prostate cancer, gastric cancer, pancreatic cancer, non-small cell lung cancer and small-cell lung cancer. The HR-proficient cancer may be ovarian cancer.

The cancer may be BRCA 1/2 proficient. The BRCA 1/2 proficient cancer may be selected from the group consisting of ovarian cancer, breast cancer, prostate cancer, gastric cancer, pancreatic cancer, non-small cell lung cancer and small-cell lung cancer. The BRCA 1/2 proficient cancer may be ovarian cancer.

The cancer may be a HR-deficient cancer. In particular, the cancer may be a HR-deficient cancer that is resistant to treatment with a DDR inhibitor, such as a PARP inhibitor. The HR-deficient cancer may be selected from the group consisting of ovarian cancer, breast cancer, prostate cancer, gastric cancer, pancreatic cancer, non-small cell lung cancer and small-cell lung cancer. The HR-deficient cancer may be ovarian cancer.

The cancer may be cancer that is resistant to treatment with a DDR inhibitor, such as a PARP inhibitor. In certain embodiments, DDR inhibitor therapy resistance, such as PARP inhibitor therapy, resistance results from homologous recombination repair restoration (HRR) in a cancer that was previously HR-deficient. Accordingly, in certain embodiments, the cancer is a cancer that was previously HR-deficient wherein PARP inhibitor therapy has resulted in homologous recombination repair restoration leading to PARP inhibitor therapy resistance.

The method may include a step of evaluating a subject suffering from cancer to identify the genetic or epigenetic makeup of cancer cells of the subject. In particular, tumors in cancer subjects may be systematically surveyed to identify the underlying somatic genetic changes in sequence, expression, and copy number. Specifically, the method may include a step of analyzing one or more tumors or cancer cells of the cancer subject for the presence of one or more deleterious mutations in genes correlated with HR-deficiency. The HRD status of the subject may be routinely tested to predict the response of the subject to treatment with a RBM39 degrader and thus assist a clinician in deciding on the best treatment for the patient.

Additionally or alternatively, the cancer may be characterized by overexpression or amplification of RNA-binding motif protein 39 (RBM39) and/or DDB1 and CUL4 associated factor 15 (DCAF15). It has been discovered, as disclosed herein, that RBM39 expression is amplified or upregulated in certain cancers (e.g., KRAS mutated cancers, acute myeloid leukemia, colon cancer, breast cancer). Additionally, as disclosed herein, DCAF15 expression is amplified or upregulated in certain cancers (e.g., neuroblastoma, hematopoietic cancers, lymphoid cancers). Accordingly, the methods disclosed herein may include a step of assessing the subject for overexpression or amplification of RNA-binding motif protein 39 (RBM39) and/or DDB1 and CUL4 associated factor 15 (DCAF15).

Cancer that is Resistant to DDR Inhibitor Therapy, Such as PARP Inhibitor Therapy

The cancer may be resistant to treatment with a DDR inhibitor, such as a PARP inhibitor. The cancer may be resistant to treatment with the DDR inhibitor, such as the PARP inhibitor, alone, that is, when the subject is treated using the DDR inhibitor as a single active agent. Additionally or alternatively, the cancer may be resistant to treatment with the DDR inhibitor, such as the PARP inhibitor, in combination with an anti-vascular endothelial growth factor (VEGF) inhibitor. The terms “resistant” or “resistance” to therapy as used herein refer to a lack of beneficial response or a reduced beneficial response to treatment with the DDR inhibitor, such as the PARP inhibitor. The subject may initially show a beneficial response to the treatment, but the beneficial response may cease or be reduced where the subject becomes resistant to the treatment. In certain aspects where the cancer is resistant to DDR inhibitor therapy, such as PARP inhibitor therapy, the cancer may be deficient in homologous recombination. In certain embodiments, DDR inhibitor therapy resistance, such as PARP inhibitor therapy, resistance results from homologous recombination repair restoration (HRR) following treatment with a DDR inhibitor, such as a PARP inhibitor.

### RNA-Binding Motif Protein 39 (RBM39) Degrader

The term RNA-Binding Motif Protein 39 (RBM39) degrader as used herein refers to compounds that may be used to target RBM39 resulting in degradation of RBM39. RBM39 degraders may act as molecular glue degraders of RBM39 by forming a ternary complex with RBM39 and the E3 ubiquitin ligase receptor DDB1 and CUL4 associated factor 15 (DCAF15) promoting the interaction of RBM39 splicing factor and CUL4-DCAF15 E3 ubiquitin ligase, leading to polyubiquitination and proteasomal degradation of RBM39.

The RBM39 degrader may be an aryl sulfonamide. The aryl sulfonamide may be indisulam, tasisulam, CQS E7820, or Compound A. Compound A has a structure of

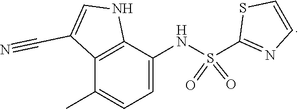 [SMILES: Cc1ccc(NS(=O)(=O)c2nccs2)c2[nH]cc(C#N)c12]

The RBM39 degrader may be E7820. The RBM39 degrader may be Compound A.

The term “RBM39 degrader” as used herein is understood to encompass pharmaceutically acceptable salts thereof. The term “RBM39 degrader” as used herein may refer to one RBM39 degrader or a combination of two or more RBM39 degraders.

### Combination Treatments

Treatment with a RBM39 degrader may be combined with one or more additional treatments for cancer. As such, the method may further comprise a step of administering an additional treatment for cancer to the subject. The RBM39 degrader thereof and the additional treatment for cancer may be administered simultaneously, sequentially or separately. The RBM39 degrader and the additional treatment for cancer may be administered in combination. The RBM39 degrader may be administered contemporaneously, previously or subsequently to the additional treatment for cancer.

The additional treatment may be any other treatment suitable for treating cancer, for example, chemotherapy or an immune checkpoint inhibitor.

In particular, the additional treatment for cancer may be a DDR inhibitor such as a PARP inhibitor, an ATR inhibitor or a CHK1 inhibitor. Accordingly, the method may further comprise a step of administering a DDR inhibitor, such as a PARP inhibitor, to the subject. The RBM39 degrader and the DDR inhibitor, such as the PARP inhibitor, may be administered simultaneously, sequentially or separately. The RBM39 degrader and the DDR inhibitor, such as the PARP inhibitor, may be administered in combination. The RBM39 degrader may be administered contemporaneously, previously or subsequently to the DDR inhibitor, such as the PARP inhibitor. The DDR inhibitor may be selected from the group consisting of inhibitors of WEE, RAD51, ATR, PolTheta, BLM, WRN, PARG, USP1 and DNAPKc.

RBM39 degraders may be used to induce a synthetic lethality phenotype when combined with DDR inhibitors, such as PARP inhibitors, thus enabling treatment of patients who would not derive benefit from treatment with the DDR inhibitor in the absence of an RBM39 degrader.

Furthermore, the RBM39 degrader and the DDR inhibitor, such as the PARP inhibitor, may have a synergistic effect in the treatment of the cancer which is greater than the additive effect of each of the RBM39 degrader and the DDR inhibitor, such as the PARP inhibitor, when administered separately. In particular, time to complete cancer regression has been shown to be reduced when RBM39 degradation is combined with PARP inhibition when compared with RBM39 degradation alone.

Treatment may be administered alone or may be administered as a pharmaceutical composition, which will generally comprise a suitable pharmaceutically acceptable excipient, diluent or carrier. The pharmaceutically acceptable excipient, diluent or carrier may be selected depending on the intended route of administration. Examples of suitable pharmaceutical carriers include water, glycerol and ethanol.

### Single Agent

The RBM39 degrader may be administered as a single agent for the treatment of the cancer. In this embodiment, an additional treatment for the cancer, for example, a PARP inhibitor, is not administered. However, treatment may still be administered as a pharmaceutical composition, which will generally comprise a suitable pharmaceutically acceptable excipient, diluent or carrier. In certain embodiments, the subject will previously have undergone treatment with a PARP inhibitor prior to commencing treatment with the RBM39 degrader.

### Composition

Also provided herein is a composition comprising a RNA-binding motif protein 39 (RBM39) degrader and a DNA repair and DNA damage response (DDR) inhibitor.

The RBM39 degrader may be an aryl sulfonamide. The aryl sulfonamide may be selected from the group indisulam, tasisulam, CQS and E7820. The RBM39 degrader may be E7820.

The DDR inhibitor may be a poly (ADP-ribose) polymerase (PARP) inhibitor. The DDR inhibitor may be an ATR inhibitor. The DDR inhibitor may be a CHK1 inhibitor. The PARP inhibitor may be selected from the group consisting of olaparib, niraparib, talazoparib and rucaparib. The PARP inhibitor may be olaparib. The PARP inhibitor may be niraparib. The DDR inhibitor may be a CHK1 inhibitor. The DDR inhibitor may be selected from the group consisting of inhibitors of WEE, RAD51, ATR, PolTheta, BLM, WRN, PARG, USP1 and DNAPKc.

The composition may comprise an aryl sulfonamide and a PARP inhibitor. For example, the composition may comprise E7820 and olaparib. The composition may comprise E7820 and niraparib. The composition may comprise indisulam and olaparib. The composition may comprise indisulam and niraparib.

The composition may be provided as a pharmaceutical composition, which will generally comprise a suitable pharmaceutically acceptable excipient, diluent or carrier. The pharmaceutically acceptable excipient, diluent or carrier may be selected depending on the intended route of administration. Examples of suitable pharmaceutical carriers include water, glycerol and ethanol. The composition or pharmaceutical composition may be used in the methods as disclosed herein.

### Subject

Typically, the terms “subject” and “patient” are used interchangeably herein. The subject is typically a mammal, more typically a human.

The subject suffering from cancer may have a cancer that is HR-proficient. The subject suffering from cancer may have a cancer that is BRCA-proficient. Alternatively, the subject suffering from cancer may have a cancer that is HR-deficient. The subject suffering from cancer may in that case have one or more deleterious mutations in one or more genes correlated with HR-deficiency in the cancer. Genes correlated with HR-deficiency may be selected from the group consisting of BRCA genes, genes related to the Fanconi Anemia repair pathway, ATM, TP53, genes related to the base excision repair pathway, genes related to the non-homologous end joining pathway and genes related to the alternative-end joining pathway. In particular, the subject may have one or more deleterious mutations in the BRCA1 and/or BRCA2 genes.

The subject may have a cancer that has developed resistance to/is resistant to a previous treatment for cancer, for example, a previous treatment with a DDR inhibitor, such as a PARP inhibitor.

The subject may have a cancer that is BRCA-proficient and has developed resistance to/is resistant to treatment with a DDR inhibitor, such as a PARP inhibitor.

Additionally or alternatively, the subject may have a cancer characterized by overexpression or amplification of RNA-binding motif protein 39 (RBM39) and/or DDB1 and CUL4 associated factor 15 (DCAF15).

The RBM39 degrader may be provided as a first line or a second line treatment. The RBM39 degrader may be provided as a second line treatment in subjects who have become resistant to a first line treatment with a DDR inhibitor, such as a PARP inhibitor.

### Administration

The RBM39 degrader and/or the DDR/PARP inhibitor may be administered in a therapeutically effective amount, this being an amount sufficient to show benefit to the subject to whom the treatment is administered. The actual dose administered, and rate and time-course of administration, will depend on, and can be determined with due reference to, the nature and severity of the condition which is being treated, as well as factors such as the age, sex and weight of the subject being treated, as well as the route of administration. Further due consideration should be given to the properties of the treatment, for example, its in-vivo plasma life and concentration in the formulation, as well as the route, site and rate of delivery. Prescription of treatment, e.g. decisions on dosage, etc., is ultimately within the responsibility and at the discretion of general practitioners and other medical doctors, and typically takes account of the disorder to be treated, the condition of the individual patient, the site of delivery, the method of administration and other factors known to practitioners.

Dosage regimens can include a single administration, or multiple administrative doses. The treatment can further be administered simultaneously, sequentially or separately with other therapeutics and medicaments that are used for the treatment of the cancer.

The treatment may be administered to a subject in need of treatment via any suitable route. In particular, the treatment may be administered systemically. The treatment may be administered orally or parenterally by injection or infusion. Examples of preferred routes for parenteral administration include, but are not limited to, intravenous, intracardial, intraarterial, intraperitoneal, intramuscular, intracavity, subcutaneous, transmucosal, inhalation and transdermal. Routes of administration may further include enteral, for example, mucosal (including pulmonary) and rectal. The treatment may be administered via nanoparticles, microspheres, liposomes, other microparticulate delivery systems or sustained release formulations placed in certain tissues including blood.

Predicting Response to Therapy with RBM39 Degraders

Further provided herein is a method of predicting a cancer subject's response to treatment with an RNA-binding motif protein 39 (RBM39) degrader.

The method may comprise determining the subject's response to treatment with a DNA repair and DNA damage response (DDR) inhibitor therapy, such as poly (ADP-ribose) polymerase (PARP) inhibitor therapy. A subject that is resistant to DDR inhibitor therapy may benefit from treatment with a RBM39 degrader.

The method may additionally or alternatively comprise using a subject's HRD status to predict their response to treatment with an RNA-binding motif protein 39 (RBM39) degrader. Accordingly, the method may comprise determining the subject's Homologous recombination deficiency (HRD) status. In particular, a subject identified as having a homologous recombination (HR)-deficient cancer may benefit from treatment with a RBM39 degrader either as a single agent or in combination with a DDR inhibitor. A subject identified as having a homologous recombination (HR)-proficient cancer may benefit from treatment with a RBM39 degrader in combination with a DDR inhibitor. As such, the disclosure provides the use of biomarkers of homologous recombination (HR)-deficient/HR-proficient cancer to evaluate the likelihood that a RBM39 degrader will produce an anti-cancer effect in a cancer subject. The anti-cancer effect may be any effect which is of benefit to treat the subject. This includes the reduction or inhibition of the progression, severity and/or duration of the cancer or at least one symptom thereof and includes curative, alleviative or prophylactic effects. The HRD/mutational status of cancer cells and/or tumors in cancer subjects may be systematically surveyed to identify the underlying somatic genetic changes in sequence, expression, and copy number, and subjects may be treated according to the genetic or epigenetic makeup of the cancer cells.

Determining the subject's HRD status may comprise quantifying loss of heterozygosity (LOH), telomeric allelic imbalance, and/or large-scale state transitions. Determining the subject's HRD status may comprise determining the presence or absence of one or more deleterious mutations in one or more genes correlated with HR-deficiency, such as breast cancer susceptibility genes 1 or 2 (BRCA1 or BRCA2), genes related to the Fanconi Anemia repair pathway, ATM, TP53, genes related to the base excision repair pathway, genes related to the non-homologous end joining pathway and genes related to the alternative-end joining pathway. In particular, determining the subject's HRD status may comprise determining the presence or absence of one or more deleterious mutations in breast cancer susceptibility genes 1 and/or 2 (BRCA1 and/or BRCA2). Additionally or alternatively, determining the subject's HRD status may comprise using one of the methods discussed above in relation to HRD status.

The method may include a step of administering an RBM39 degrader to the subject.

The method may also include a step of administering a DNA repair and DNA damage response (DDR) inhibitor, such as a Poly (ADP-Ribose) Polymerase (PARP) inhibitor, to the subject.

An RBM39 degrader may be administered where the subject is identified as having a homologous recombination (HR)-deficient cancer. An RBM39 degrader may be administered where the subject is identified as having a homologous recombination (HR)-proficient cancer. An RBM39 degrader may be administered where the subject is identified as having a BRCA-proficient cancer.

A subject identified as having a HR-deficient cancer that is resistant to treatment with a DDR inhibitor, such as a PARP inhibitor, may also benefit from treatment with a RBM39 degrader. Accordingly, where the subject's HRD status is determined as HR-deficient, the method may include a step of determining the subject's response to treatment with a DNA repair and DNA damage response (DDR) inhibitor, such as a PARP inhibitor, wherein a subject with a HR-deficient cancer that is resistant to treatment with a DDR inhibitor, such as a PARP inhibitor, may benefit from treatment with a RBM39 degrader. The method may include a step of administering an RBM39 degrader to the subject wherein the subject is identified as having a HR-deficient cancer and being resistant to treatment with the PARP inhibitor. The method may also include a step of administering a DDR inhibitor, such as a PARP inhibitor, to the subject.

Additionally or alternatively, the method may include a step of assessing the cancer for overexpression and/or amplification of RNA-binding motif protein 39 (RBM39) and/or DDB1 and CUL4 associated factor 15 (DCAF15) wherein a subject with overexpression and/or amplification of RBM39 and/or DCAF15 may benefit from treatment with a RBM39 degrader. The method may therefore include a step of administering an RBM39 degrader to the subject wherein the subject is identified as having overexpression or amplification of RBM39 and/or DCAF15.

### Definitions

Unless otherwise defined, all technical and scientific terms used herein have the meaning commonly understood by a person who is skilled in the art in the field of the present disclosure.

The term “treatment” as used herein and associated terms such as “treat” and “treating” means the reduction or inhibition of the progression, severity and/or duration of cancer or at least one symptom thereof. The term ‘treatment’ therefore refers to any regimen that can benefit a subject. Treatment may include curative, alleviative or prophylactic effects.

It is appreciated that certain features of the invention, which are, for clarity, described in the context of separate embodiments, may also be provided in combination in a single embodiment. Conversely, various features of the invention that are, for brevity, described in the context of a single embodiment, may also be provided separately or in any suitable sub-combination.

### EXAMPLES

### Example 1—Efficacy of RBM39 Degrader E7820 Alone and in Combination with PARP Inhibitor Olaparib in the HRD Negative High-Grade Serous Ovarian Cancer OVCAR3 CDX Model

### Materials and Methods

Cell culture: OVCAR3 is a human ovarian cancer cell line that is BRCA-proficient and HRD negative. Crown biosciences in China acquired this cell line for in vivo testing. OVCAR3 human ovarian tumor cells were maintained in vitro with RPMI1640 supplemented with 20% fetal bovine serum and 10 μg/ml insulin at 37° C. in an atmosphere of 5% CO2 in air. The cells in exponential growth phase were harvested and quantitated by cell counter before tumor inoculation.
Tumor Inoculation: Each mouse was inoculated subcutaneously in the right upper flank region OVCAR-3 tumor cells (1×10⁷ cells) with matrigel in 0.1 ml of PBS for tumor development.
Randomization: The randomization started from when the mean tumor size reached approximately 88 mm³. BALBc/nude mice were enrolled in the study. All animals were randomly allocated to 7 study groups. Randomization was performed based on “Matched distribution” method. The date of randomization was denoted as day 0.
Tumor Growth Inhibition (TGI): Tumor Growth Inhibition (TGI): Methods of TGI are known in the art. Tumor volumes were measured three per week after randomization in two dimensions using a caliper, and the volume was expressed in mm³ using the formula: V=(L×W×W)/2, where Vis tumor volume, L is tumor length (the longest tumor dimension) and W is tumor width (the longest tumor dimension perpendicular to L). % TGI was calculated using the formula % TGI=(TV vehicle−TV treatment)/(TV vehicle−TV initial)*100 for all mice (Wong, H, et al. Clin Cancer Res., 2012, 18(14): 3846-3855)
Treatment Arms: 10 mice per arm were treated with either Vehicle, olaparib, E7820, E7820 plus olaparib, or paclitaxel for 21 days. Olaparib was administered PO QD at 100 mg/kg, E7820 was administered PO BID at 50 mg/kg, and paclitaxel was administered IV QW at 15 mg/kg.

### Results

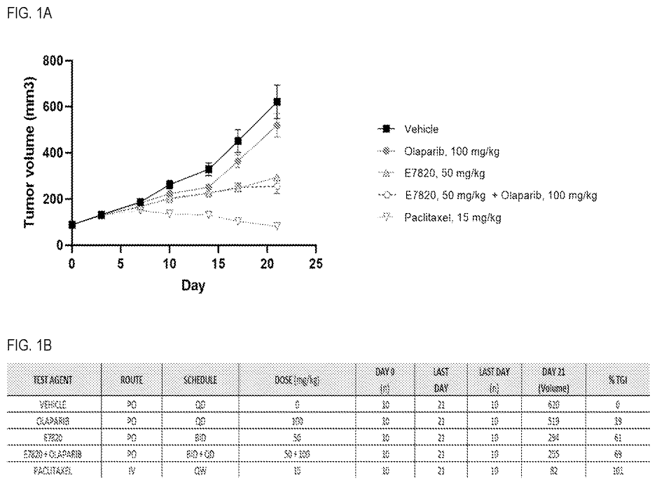

The results are shown in FIG. 1A (tumor volume) and FIG. 1B (Tumor Growth Inhibition (TGI)). FIG. 1A shows that tumor volume decreased with treatment with single agent E7820 at 50 mg/kg and the combination of E7820 at 50 mg/kg plus olaparib 100 mg/kg in the HRD negative OVCAR3 CDX model and was resistant to treatment with olaparib. RBM39 degradation alone or in combination with PARP inhibition was therefore superior to treatment with PARP inhibition alone in the HRD negative model.

### Example 2—Immunohistochemistry (IHC) Protein Expression Analysis for DNA Damage Response Proteins and Cell Cycle Checkpoint Proteins on Tumor Samples in Example 1

### Materials and Methods

Sample Collection: 16 tumor samples were collected across the vehicle, olaparib, E7820 single agent, and paclitaxel arms from the in vivo OVCAR3 CDX study described in Example 1

### Primary Antibodies:

| Antibody | Company | Cat# | Type | Reactivity |
|---|---|---|---|---|
| Phospho- | Cell | 9701S | Rabbit | Human, |
| Histone | Signaling | | IgG mAb | Mouse, Rat, |
| H3 (Ser10) | | | | Monkey, |
| Antibody | | | | Xenopus |
| Anti-MCL1 | Abcam | ab32087 | Rabbit | Mouse, Rat, |
| Antibody | | | IgG mAb | Human |
| [Y37] | | | | |
| Anti-BRCA1 | Abcam | Ab213929 | Rabbit | Human |
| Antibody | | | IgG mAb | |
| [EPR19433] | | | | |
| Anti-CDK12 | Abcam | ab246887 | Rabbit | Human |
| Antibody | | | IgG mAb | |
| Anti-RBM39 | Atlas | HPA001591 | Rabbit | Human |
| Antibody | Antibodies | | IgG mAb | |
| Anti-Cdk1-2-3-5 | Abcam | ab206314 | Rabbit | Human |
| antibody | | | IgG mAb | |
| [EP762RY] | | | | |
| Recombinant | Abcam | ab243863 | Rabbit | Human |
| Anti-Cdk7 | | | IgG mAb | |
| antibody | | | | |
| [BL-80-3D4] | | | | |
| Recombinant | Abcam | ab75848 | Rabbit | Human |
| Anti-Cdk9 | | | IgG mAb | |
| antibody | | | | |
| [EP3118] | | | | |

### Secondary Antibody Detection Kit

| Antibody | Company | Catalog | Detailed information |
|---|---|---|---|
| Bond Polymer | Leica | DS9800 | Anti-rabbit Poly-HRP-IgG |
| Refine | | | (<25 μg/mL) containing |
| Detection | | | 10% (v/v) animal serum |
| | | | in tris-buffered saline/ |
| | | | 0.09% ProClin ™ 950 |
| | | | (ready-to-use) |

### Isotype

| Antibody | Company | Catalog | Concentration | Dilution |
|---|---|---|---|---|
| Rabbit IgG, | Vector | I-1000 | 1 ug/uL | Diluted at the same |
| polyclonal | | | | concentration with |
| | | | | primary antibody |

### Tissue Processing

1) Collect fresh specimens and place in 10% NBF (neutral-buffered formalin; fixative volume/tissue, 10˜20 folds), fix at room temperature for 24 hours.
2) Trim the fixed tissue at the thickness of 3-5 mm.
3) Move the tissue into the Embedding box, and snap the box into deionized water for 30 minutes, change water twice every 30 minutes.
4) After washing, the fixed tissues are transferred to the LEICA ASP300S Vacuum Tissue Processor for dehydration.

### Dehydration Procedure

### Automatized Dehydration Procedure

| Reagent | Duration | Temperature ° C. | Pressure/Vacuum |
|---|---|---|---|
| 70% ethanol | 20 min* | RT | — |
| 80% ethanol | 20 min | RT | — |
| 90% ethanol | 20 min | RT | — |
| 95% ethanol | 25 min | RT | on |
| 100% ethanol | 25 min | RT | — |
| 100% ethanol | 35 min | RT | — |
| 100% ethanol | 35 min | RT | on |
| Xylene | 35 min | RT | — |
| Xylene | 35 min | RT | on |
| Paraffin I | 30 min | 60 | — |
| Paraffin II | 30 min | 60 | — |
| Paraffin III | 30 min | 60 | on |

*Note:
The pressure and vacuum will be in cycle operation when turns on “Pressure/Vacuum”. The fluid will be output when it pressures and the fluid goes back when it is vacuum.
*The duration time of the first step can be moderately extended and will not affect final results.

### FFPE Blocks Preparation

Samples followed by standard embedding process to form FFPE block. Tissues are embedded in paraffin on Paraffin Embedding Station. The mouse ID number is included on the sample label with the other information.

### FFPE Slides Preparation

FFPE blocks are sectioned with a manual rotary microtome, 4 μm thickness/section. The slide label for each sample contains all the necessary information, including project code, target name, group number, tissue type, etc.

### IHC Procedures on Bond RX Autostainer

| | | Time (minute) | |
|---|---|---|---|
| Step | Reagent | per cycle | Temperature(° C.) |
| 1 | Dewax solution | 0.5 | 72 |
| 2 | Dewax solution | 0 | 72 |
| 3 | Dewax solution | 0 | RT |
| 4 | Alcohol | 0 | RT |
| 5 | Alcohol | 0 | RT |
| 6 | Alcohol | 0 | RT |
| 7 | Bond Wash buffer | 0 | RT |
| 8 | Bond Wash buffer | 0 | RT |
| 9 | Bond Wash buffer | 0 | RT |
| 10 | Bond ER1/ER2 | 0 | RT |
| 11 | Bond ER1/ER2 | 0 | RT |
| 12 | Bond ER1/ER2 | 20 | 100 |
| 13 | Bond ER1/ER2 | 0 | RT |
| 14 | Bond Wash buffer | 0 | RT |
| 15 | Bond Wash buffer | 0 | RT |
| 16 | Bond Wash buffer | 0 | RT |
| 17 | Bond Wash buffer | 0 | RT |
| 18 | Bond Wash buffer | 3 | RT |
| 19 | Peroxide Block | 10 | RT |
| 20 | Bond Wash buffer | 0 | RT |
| 21 | Bond Wash buffer | 0 | RT |
| 22 | Bond Wash buffer | 0 | RT |
| 23 | Primary antibody 1 | 60 | RT |
| 24 | Bond Wash buffer | 0 | RT |
| 25 | Bond Wash buffer | 2 | RT |
| 26 | Bond Wash buffer | 2 | RT |
| 27 | Bond Wash buffer | 2 | RT |
| 28 | Secondary antibody | 20 | RT |
| 29 | Bond Wash buffer | 0 | RT |
| 30 | Bond Wash buffer | 2 | RT |
| 31 | Bond Wash buffer | 2 | RT |
| 32 | Bond Wash buffer | 2 | RT |
| 33 | Deionized Water | 0 | RT |
| 34 | DAB Refine | 0 | RT |
| 35 | DAB Refine | 5 | RT |
| 36 | Deionized Water | 0 | RT |
| 37 | Deionized Water | 0 | RT |
| 38 | Deionized Water | 0 | RT |
| 39 | Hematoxylin | 10 | RT |
| 40 | Deionized Water | 0 | RT |
| 41 | Bond Wash buffer | 0 | RT |

*Note:
RT: room temperature; ER1: Epitope retrieval solution 1: (Citrate buffer (pH 6.0)) for antigen retrieval; ER2: Epitope retrieval solution 2: (EDTA buffer (pH 9.0) for antigen retrieval; Time = 0 means that the slide will be rinsed with the solution very quickly; Mount the sections with SlowFade ® Gold Anti-fade Mountant, Cat#S36938, Invitrogen.

### Data Analysis

All stained sections are scanned with NanoZoomer-HT 2.0 Image system for 40× magnification. High resolution picture for whole section is generated and further quantification analyzed by HALO. All the scanned images are analyzed by HALO™ platform. The whole slide image is analyzed and necrosis and stroma area are excluded, only viable tumor area is scored. The intensity of specific staining is scored at four levels, 0 (negative), 1+(weak staining), 2+(medium staining), 3+(strong staining). The percentages of tumor cells at different intensity levels are evaluated with H-score. H-Score=(% at 0)×0+(% at 1)×1+(% at 2)×2+(% at 3)×3 (H-Score range is 0 to 300).

### Results

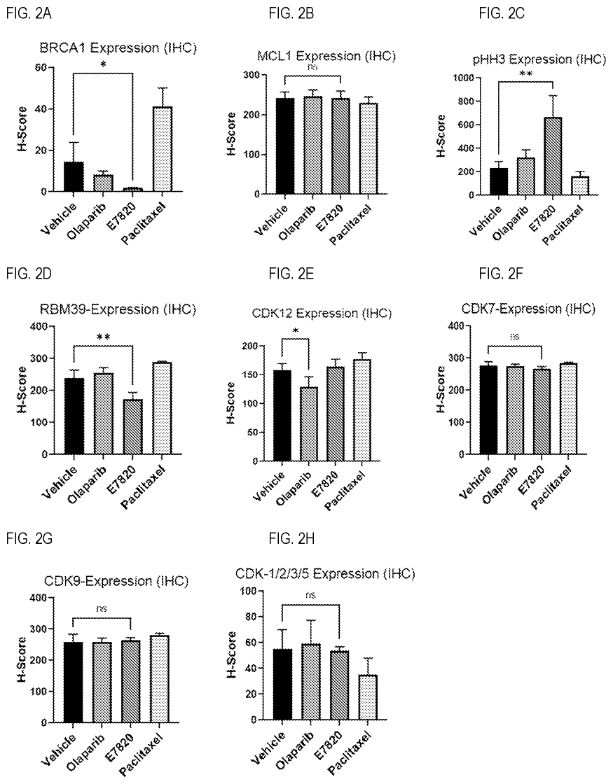

The results are shown in FIG. 2A-H and illustrate that E7280 can sufficiently downregulate BRCA1 protein expression (FIG. 2A) and upregulate pHH3 protein expression in OVCAR3 tumor samples (FIG. 2C) while no significant changes are seen with olaparib treatment. This provides evidence that E7820 can directly impact Homologous recombination machinery. Additionally, no changes in the protein expression for MCL1, CDK7, CDK9, CDK12, and CDK 1/2/3/5 were seen with E7820. This data provides evidence that RBM39 degradation can decrease DDR repair machinery as well as promote cell cycle progression by overriding the G2/M checkpoint and pushing cells into mitosis unrepaired, without globally impacting cyclin dependent kinase protein functions.

### Example 3—Efficacy of RBM39 Degrader E7820 Alone and in Combination with PARP Inhibitor Olaparib in the BRCA-Proficient Ovarian Cancer OV0273 PDX Model

### Materials and Methods

Tumor Inoculation: Study was conducted at Crown Biosciences in China. Tumor fragments from stock mice were harvested and used for inoculation into mice. Each mouse was inoculated subcutaneously in the right upper flank with primary human ovarian tumor xenograft model OV0273 tumor fragment (2-3 mm in diameter) for tumor development.
Randomization: The randomization started from when the mean tumor size reaches approximately 149 mm³ and female 40 BALB/c Nude mice were enrolled in the study. All animals were randomly allocated to 4 study groups. Randomization was performed based on “Matched distribution” method. The date of randomization was denoted as day 0.
Tumor Growth Inhibition (TGI): Tumor Growth Inhibition (TGI): Methods of TGI are known in the art. Tumor volumes were measured three per week after randomization in two dimensions using a caliper, and the volume was expressed in mm³ using the formula: V=(L×W×W)/2, where Vis tumor volume, L is tumor length (the longest tumor dimension) and W is tumor width (the longest tumor dimension perpendicular to L). % TGI was calculated using the formula % TGI=(TV vehicle−TV treatment)/(TV vehicle−TV initial)*100 for all mice (Wong, H, et al. Clin Cancer Res., 2012, 18(14): 3846-3855)
Treatment Arms: 10 mice per arm were treated with either Vehicle, olaparib, E7820, or E7820 plus olaparib, for 28 days. Olaparib was administered PO QD at 90 mg/kg, E7820 was administered PO BID at 85 mg/kg. Vehicle formulation matched E7820 and followed prior publications. BID timepoint for E7820 was 12 hours.

### Results

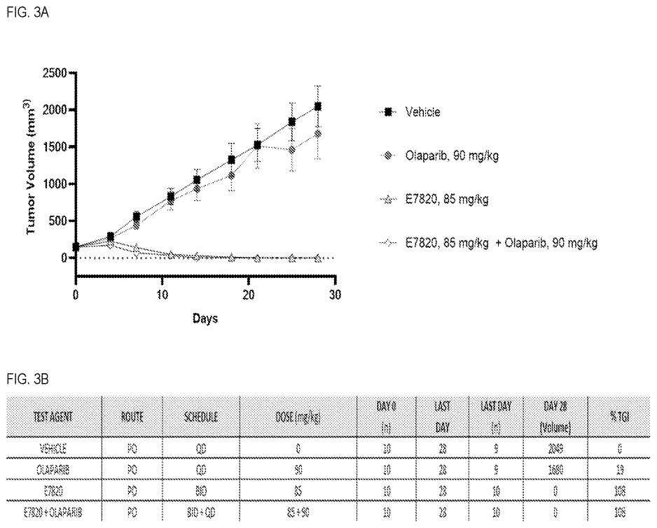

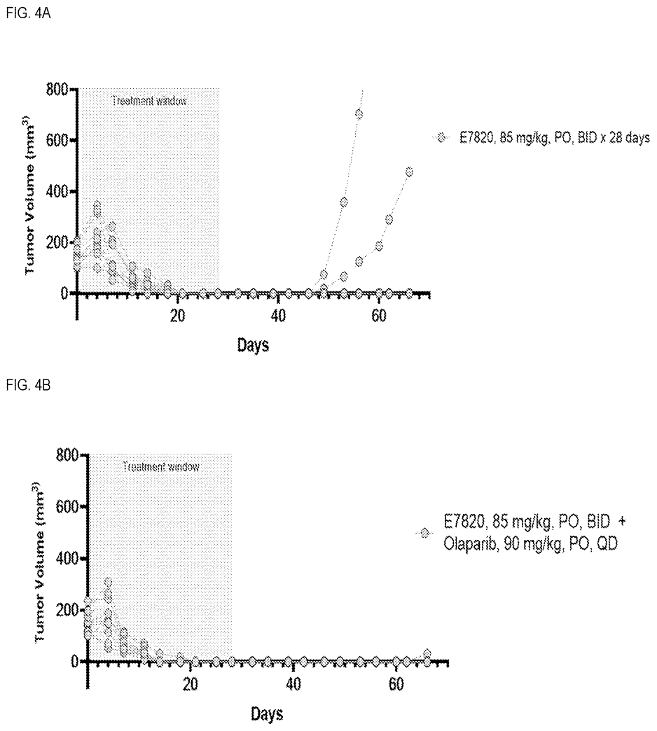

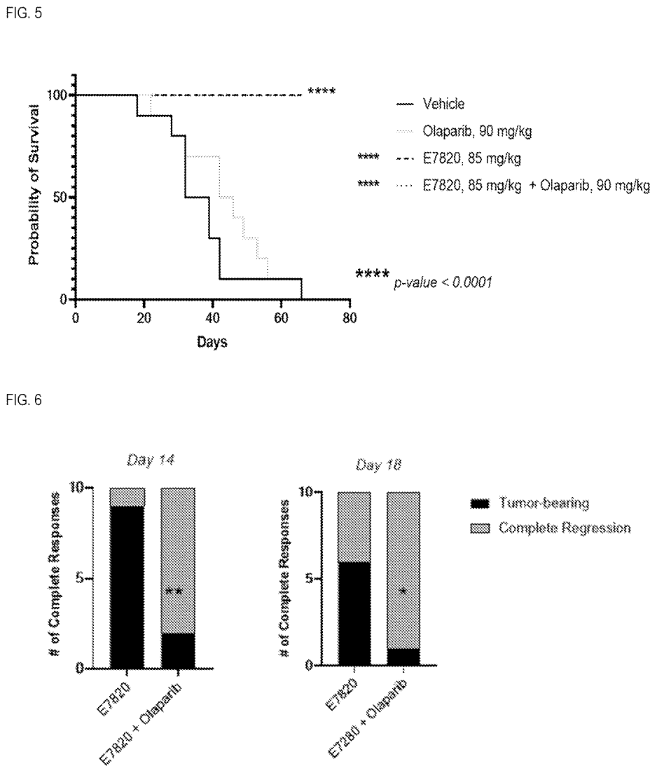

The results are shown in FIG. 3A (tumor volume) and FIG. 3B (Tumor Growth Inhibition (TGI)). FIG. 3A shows that tumor volume decreased with treatment with single agent E7820 at 85 mg/kg and the combination of E7820 at 85 mg/kg plus olaparib 90 mg/kg in the BRCA-proficient OV0273 PDX model and was resistant to treatment with olaparib. RBM39 degradation alone or in combination with PARP inhibition was therefore superior to treatment with PARP inhibition alone in the BRCA-proficient ovarian PDX mouse model.
The results of tumor growth curves for individual mice are shown in FIG. 4A (E7820 monotherapy) and FIG. 4B (combination of E7820 plus olaparib). FIGS. 4A and 4B both demonstrate that all mice continue to maintain complete regressions after 28 days of treatment with two mice displaying re-growth of tumor in the E7820 at 85 mg/kg single agent arm alone starting around day 48 while 0 mice in the combination arm of E7820 at 85 mg/kg plus olaparib at 90 mg/kg remain tumor-free at the same day 48 timepoint. RBM39 degradation alone or in combination with PARP inhibition is sufficient to produce robust durable responses in the BRCA-proficient ovarian PDX mouse model.
Survival analysis of each group in the study is shown in FIG. 5. Single agent E7820 at 85 mg/kg and the combination of E7820 at 85 mg/kg plus olaparib 90 mg/kg in the BRCA-proficient OV0273 PDX model prolonged median survival time >70 days compared with vehicle control group and olaparib group, with a statistically significant difference observed (p<0.0001). Kaplan-Meier analysis was tested for significance using log-rank test. RBM39 degradation alone or in combination with PARP inhibition is sufficient to produce survival benefit in the BRCA-proficient ovarian PDX mouse model.
Time to complete regression results are shown in FIG. 6. At day 14, 10% of mice (n=1) treated with single agent E7280 at 85 mg/kg achieved complete regression while 80% of mice (n=8) treated with the combination of E7820 at 85 mg/kg plus olaparib at 90 mg/kg in the BRCA-proficient ovarian PDX mouse model, with a statistically significant difference observed (p<0.01) between the combination group and the single agent group as determined using a Chi-Square test to determine the difference in proportions between groups. RBM39 degradation in combination with PARP inhibition was therefore superior in achieving a faster time to complete regression than observed with RBM39 degradation alone suggesting a synergistic effect in combination.

### Example 4—Efficacy of RBM39 Degrader E7820 Alone and in Combination with PARP Inhibitor Olaparib in the BRCA-Proficient Ovarian Cancer OV90 CDX Model

### Materials and Methods

Cell culture: OV90 is a human ovarian high-grade serous ovarian cancer cell line that is BRCA-proficient. LabCorp acquired this cell line from ATCC for in-vivo testing. OV90 human ovarian tumor cells were maintained in vitro in DMEM, 10% NHI FBS, and 1% PSG.
Tumor Inoculation: NSG female mice were inoculated subcutaneously in the right axilla region with serum-free DMEM OV90 tumor cells (1×10\hat{ }⁶ trypan-excluding cells) for tumor development.
Randomization: All mice were sorted into study groups based on caliper estimation of tumor burden. The mice were distributed to ensure that the mean tumor burden for all groups was within 10% of the overall mean tumor burden for the study population. The mean estimated tumor burden for all groups in the experiment on the first day of
treatment was 123 mm³. All animals weighed at least 14.9 g at the initiation of therapy with an overall mean body weight of 20.9 g. Tumor burden and body weights for all groups in the experiment were well-matched (within 10% of overall mean).
Tumor Growth Inhibition (TGI): Tumor Growth Inhibition (TGI): Methods of TGI are known in the art. Tumor volumes were measured three per week after randomization in two dimensions using a caliper, and the volume was expressed in mm³ using the formula: V=(L×W×W)/2, where Vis tumor volume, L is tumor length (the longest tumor dimension) and W is tumor width (the longest tumor dimension perpendicular to L). % TGI was calculated using the formula % TGI=(TV vehicle−TV treatment)/(TV vehicle−TV initial)*100 for all mice (Wong, H, et al. Clin Cancer Res., 2012, 18(14): 3846-3855)
Treatment Arms: 10 mice per arm were treated with either Vehicle, niraparib, olaparib, E7820, E7820 plus niraparib, or E7820 plus olaparib for 21 days. Niraparib was administered PO QD at 40 mg/kg, olaparib was administered PO QD at 100 mg/kg, and E7820 was administered PO BID at 50 mg/kg,

### Results

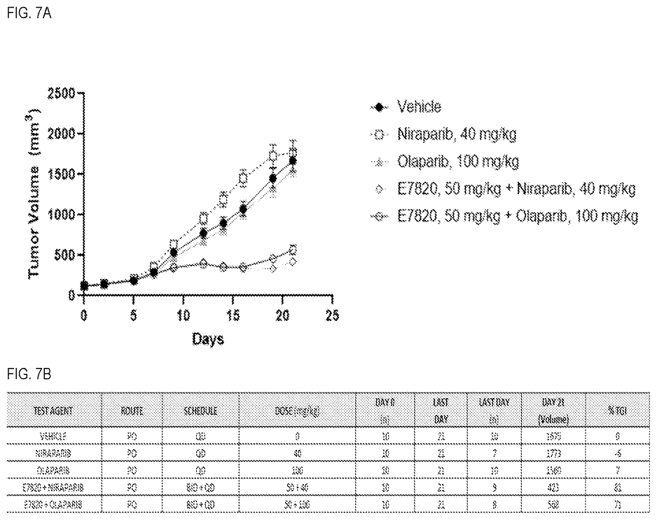

The results are shown in FIG. 7A (tumor volume) and FIG. 7B (Tumor Growth Inhibition (TGI)). FIG. 7A shows that tumor volume decreased with treatment with the combination of Niraparib at 40 mg/kg plus E7820 at 50 mg/kg and the combination of olaparib at 100 mg/kg plus E7820 at 50 mg/kg in the BRCA-proficient OV90 CDX model compared both the single agent niraparib group and olaparib group and was resistant to treatment with both single agent niraparib and olaparib groups, respectively, with a statistically significant difference observed (p<0.0001) as analyzed using 2way ANOVA. RBM39 degradation in combination with PARP inhibition was therefore superior to treatment with PARP inhibition alone in the BRCA-proficient and PARP inhibitor resistant ovarian CDX mouse model.

### Example 5—CellTiter-Glo Assay for Assessing Impact of RBM39 Degraders on Cell Viability & Proliferation In Vitro in OVCAR3 Cell Line

### Materials and Methods

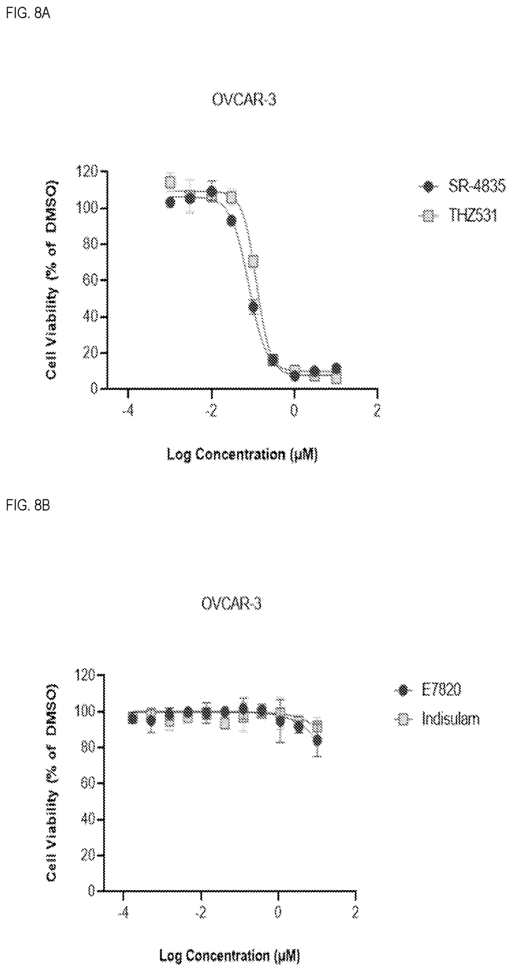

### FIG. 8A:

Cell culture: OVCAR3 is a human ovarian cancer cell line that is BRCA-proficient and HRD negative. Pharmaron in China acquired this cell line for in vitro testing. OVCAR3 human ovarian tumor cells were maintained in vitro with RPMI-1640 (Glutamax)+0.01 mg/ml insulin+20% FBS+1% PS
Cell Seeding: Cells were harvested from flask into cell culture medium and counted. Cells were diluted with culture medium for below cell densities and 8000 OVCAR cells were plated per well in a 384-well plate (PerkinElmer, Cat. #6007680), using electronic multichannel pipette in 40 μL. For the low control wells, 40 μL PBS was added. For the Plates were covered with lid and spun at 1 min/1000 rpm followed by incubation overnight at 37° C., 5% CO2.
Compound Time: SR4835 and THZ531 were dissolved at 10 mM stock solution and diluted in a 3-fold, 9-point diluted series. Cells were treated with 40 nL of SR4835 and THZ531 by plate reformat Echo550 followed by incubation at 37° C. for 72 h in a CO₂ incubator. For the high control, 40 nL DMSO was added.
Readout: CellTiter-Glo@ 2.0 Reagent (Promega, G9242) was removed from the fridge and equilibrated at RT(23° C.) for 60 minutes. Plates were removed from the incubator and allowed to equilibrate to room temperature, for at least 15 minutes. 30 μL CellTiter-Glo® 2.0 Reagent was added to the assay plates and allowed to sit for 30 minutes prior to reading. Luminescence signal was read on Envision.
Calculation: Cell viability (% DMSO control) was calculated using the following equation

$$100 - 100 * ( High control luminescence - luminescence compound ) / ( High control luminescence - Low control luminescence )$$

$$High control = 0.1 \% DMSO$$

$$Low control = PBS wells$$

### FIG. 8B:

Cell culture: OVCAR3 is a human ovarian cancer cell line that is BRCA-proficient and HRD negative. Pharmaron in China acquired this cell line for in vitro testing. OVCAR3 human ovarian tumor cells were maintained in vitro with RPMI-1640 (Glutamax)+0.01 mg/ml insulin+20% FBS.
Cell Seeding: Cells were harvested from flask into cell culture medium and counted. Cells were diluted with culture medium for below cell densities and 8000 OVCAR cells were plated per well in a 384-well plate (PerkinElmer, Cat. #6007680), using electronic multichannel pipette in 50 μL For the low control wells, 50 μL PBS was added. Plates were covered with lid and spun at 1 min/1000 rpm followed by incubation overnight at 37° C., 5% CO2.
Compound Time: E7820 and indisulam were dissolved at 10 mM stock solution and 50 nL was transferred to 10 μL of diluted solution to a 384 LDV-plate (LABCYTE, LP-0200) in a 3-fold, 11-point diluted series. For high control wells, 50 nL DMSO was added. Cells were treated with E7820 and indisulam by plate reformat Echo software and spun 1 min/1000 rpm followed by incubation at 37° C. for 72 h in a CO₂ incubator.
Readout: CellTiter-Glo@ 2.0 Reagent (Promega, G9242) was removed from the fridge and equilibrated at RT (23° C.) for 60 minutes. Plates were removed from the incubator and allowed to equilibrate to room temperature, for at least 30 minutes. 40 μL CellTiter-Glo® 2.0 Reagent was added to the assay plates, and spun 1 min/1000 rpm, then allowed to sit for 20 minutes prior to reading. Luminescence signal was read on Envision.
Calculation: Cell viability (% DMSO control) was calculated using the following equation

$$100 - 100 * ( High control luminescence - luminescence compound ) / ( High control luminescence - Low control luminescence )$$

$$High control = 0.1 \% DMSO$$

$$Low control = PBS wells$$

### Results

The cell viability results are shown with CDK12/13 inhibitors THZ531 and SR4835 in FIG. 8A and RBM39 degraders E7820 and indisulam in FIG. 8B. FIG. 8A shows that OVCAR3 cells treated with THZ531 or SR4835 lead to dose-dependent decreases in cell viability. FIG. 8B shows that OVCAR3 cells treated with E7820 or indisulam had minimal effect on cell viability. RBM39 degradation does not impact cell viability in an HRD negative ovarian cancer cell line while CDK12/13 inhibition demonstrates dose-dependent decreases in cell viability. RBM39 degradation therefore is considerably less cytotoxic to OVCAR3 cells as compared to CDK12/13 inhibition when treated in-vitro.

### Example 6—NanoBRET Target Engagement Intracellular Kinase Assay for Assessing Impact of RBM39 Degraders on CDK12+Cyclin K Kinase Activity

### Materials and Methods

Cell culture: CDK12 NanoBRET assay kit was obtained from Reaction Biology with experiment performed by Pharmaron. HEK293 cells were cultured using 90% DMEM with 10% FBS. Cells were cultivated in T-75 flasks in a cell culture incubator set at 37° C., 5% CO², 95% relative humidity. Once cells reached 80-90% confluence, they were detached and split. Cultivated cells in T-75 flasks were rinsed with 5 mL PB and aspirated off. 1.5 mL trypsin was added and incubated at 37° C. for approximately 5 minutes or until the cells detached and started to float. Trypsin was inactivated by adding excess serum containing medium.
Transient Transfection of HEK293 Cells: Cell culture medium was removed from cell flask by aspiration and trypsinized, allowing cells to dissociate from flask. Trypsin was neutralized using cell culture medium and centrifuged at 200×g for 5 minutes to pellet cells. Medium was aspirated and resuspended in assay medium consisting of 99% Opti-MEM, reduced serum medium with no phenol red and 1% FBS. Density was adjusted to 2×10⁵ cells/ml using assay medium. 10 μg/ml solution of DNA in Opti-MEM was prepared consisting of 9 μg/mL of CCNK, 1 ug/mL of Kinase-NanoLuc fusion vector DNA CDK12, and 1 mL of Opti-MEM. This solution was mixed thoroughly. 30 μl of FuGENE HD Transfection Reagent was added into each milliliter of DNA mixture to form lipid: DNA complex. Mixture was inverted 5-10 times and incubated at ambient temperature for 20 minutes to allow complexes to form. In a sterile, conical tub, 1 part of lipid DNA complex (1 mL) was mixed with 20 parts of HEK293 cells (20 mL) in suspension at 2×10⁵ cells/mL. Tube was mixed gently by inversion 5 times. 40 μL cells+lipid: DNA complex were then dispensed into a sterile tissue-culture treated 384-well assay plate and incubated 20-30 hours.
NanoBRET Tracer Reagent Addition to Cells. 5 compounds (E7820, indisulum, THZ531, CR8 and AT7519) were diluted with DMSO at 10 μM in 3-fold dilutions with 10 points each. 40 nL of compound at each concentration was transferred into the 384 well plate by Echo. 400 μM of the NanoBRET Tracer Reagent was prepared and 100 nL of tracer was added to 384 well plate. Plate was then incubated at 37° C., 5% CO² for 2 hours. 3× Complete Substrate plus Inhibitor Solution in Opti-MEM® I Reduced Serum Medium, no phenol red was prepared as follows: 48 μL NanoBRET Nano-Glo Substrate; 16 μL extracellular NanoLuc Inhibitor; 7936 μL Opti-MEM reduced serum medium, no phenol red. Lastly, 20 μL of this 3× complete substrate plus inhibitor solution was added to each well of the 384-well plate and incubated for 2-3 minutes at room temperature and read the plate on the Envision.
Calculation: BRET Ratio was calculated as follows:

$$BRET Ratio = Acceptor sample / Donor sample \times 1000$$

$$High control = Cell + DMSO + Tracer$$

$$Low control = Cell + 30 uM AT 7519 + Tracer$$

### Results

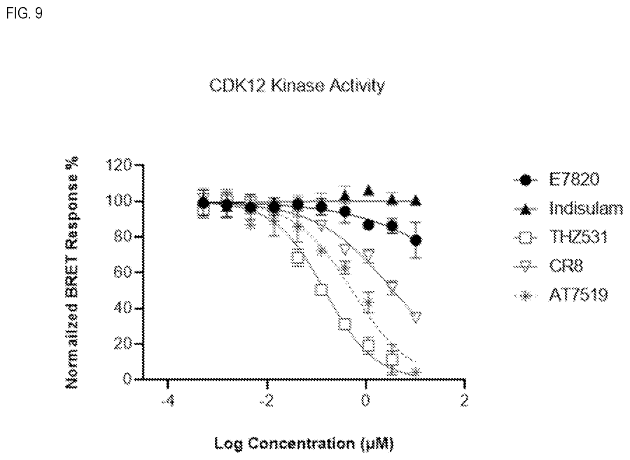

The CDK 12 kinase activity results are shown with CDK12/13 inhibitor THZ531, CDK12 degrader CR8, RBM39 degraders E7820 and indisulam, and the pan-CDK inhibitor AT7519 in FIG. 9. RBM39 degradation does not affect CDK12 kinase activity while CDK12/13 inhibition demonstrates dose-dependent inhibition of kinase function. RBM39 degradation therefore has no impact on CDK12 activity as compared to CDK12/13 inhibition or CDK12 degradation in-vitro.

### Example 7—DDR Gene Expression Analysis in OVCAR3 Cells Treated with the RBM39 Degrader E7820

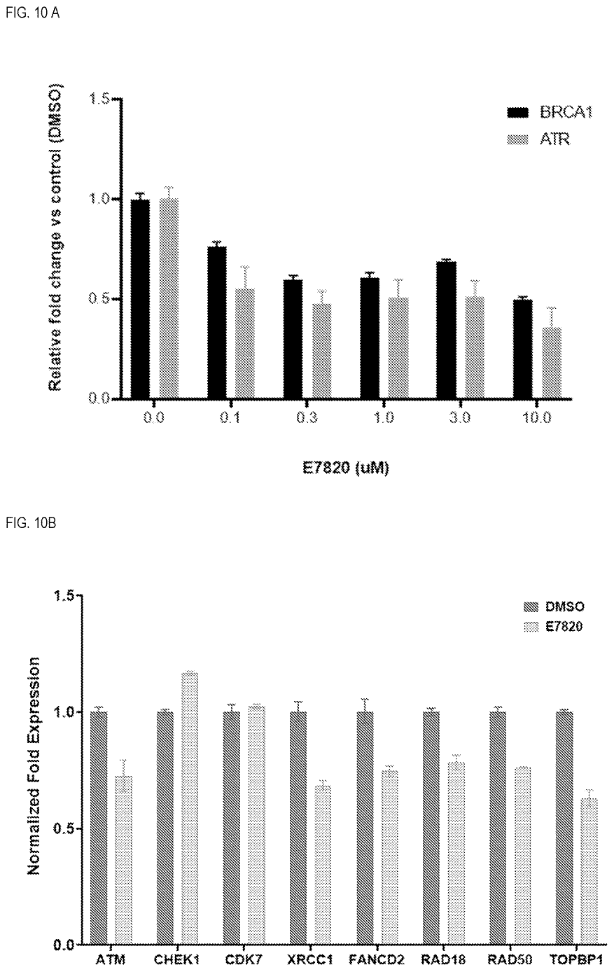

### FIG. 10A:

### Materials and Methods

Cell Seeding: OVCAR3 cells from flask were harvested into cell culture medium and then cell number was counted. Cells were diluted with culture medium for 1e6 cells/well into each well of 6-well cell culture plate. Plates were covered with lid and placed in room temperature for 30 minutes after gently shaking and then transferred into 37° C. 5% CO₂ incubator overnight for cell attachment.
Compound Treatment: E7820 was dissolved at 10 mM DMSO stock solution and diluted 3-fold for 5 doses with 10 mM as the top concentration. Diluted series were added in 1/1000 dilution and treated for 6 hours. 6 replicates per concentration. Cells pellet were collected with total RNA extracted
RNA purification: Purify RNA using the PureLink RNA Mini Kit (Invitrogen, 12183018A). 300 μL lysis buffer with 1%-2%-mercaptoethanol was added to the sample to lyse the cells for 20 minutes on ice. Lysates were centrifuged at 12000 rpm for 15 minutes and supernatant was transferred to a clean RNase-free tube. One volume 70% ethanol to each volume of cell lysate was added and vortexed. Up to 700 μL of sample was transferred to the spin cartridge and centrifuged at 12000 g for 15 sec at RT. Flow-through was discarded and repeated until entire samples were processed. 700 μL wash buffer I was added to the spin cartridge and centrifuged at 12000 g for 15 sec at RT. Flow through was discarded. Next, 500 μL wash buffer II with ethanol was added to the spin cartridge and centrifuged at 12000 g for 15 sec at RT. Flow-through was discarded and repeated. After washing, spin cartridge was centrifuged at 12000 g for 2 min to dry the membrane with bound RNA. 30 μL was RNAse-free water was added to the center of the spin cartridge and incubated for 1 min at RT. Spin cartridge was then centrifuged for 2 min to elute the RNA from the membrane into recovery tube. Purified RNA was then stored
Calculation: concentration of RNA was quantitated using Nano-Drop. Reverse transcription of RNA to cDNA done by High-Capacity RNA-to-cDNA Kit (Invitrogen, 4387406). 3 housekeeping genes were used as reference (ACTB, GAPDH, 18S). ACT was calculated by using following equation: ΔCT=CT_{Target}−CT_{Reference genes} for E7280 from 0 uM to 10 μM top concentration. Relative mRNA expression (ΔΔCT) was calculated as ΔΔCT=2^{-ΔCT} Relative expression (folds to DMSO) was calculated with error bar of plot shown as SEM.

### FIG. 10B:

### Materials and Methods

### 1. Reagents for In-Vitro Analysis

| Regents | Vendor | Cat# |
|---|---|---|
| RPMI 1640 Culture Medium | Gibco | 11875-093 |
| Insulin | Invitrogen | 12585-014 |
| Trypsin-EDTA solution | Solarbio | T1300 |
| Penicillin-Streptomycin Liquid | Solarbio | P1400 |
| PBS | Solarbio | P1020 |
| DMSO | Merck | 1029312500 |
| FBS | Sigma | F8687 |
| TaqMan fast Advanced | Invitrogen | A35377 |
| Cells-to-Ct kit | | |

### 2. Consumables for In-Vitro Analysis

| Consumables | Vendor | Cat# |
|---|---|---|
| 6-Well Culture Plate | Corning | 3516 |
| Countess cell counting chamber | Invitrogen | C10283 |
| T225 cell culture flask | Corning | 431082 |
| 50 ml polypropylene | BD-Falcon | 352098 |
| centrifuge tube | | |
| 15 ml polypropylene | BD-Falcon | 352097 |
| centrifuge tube | | |
| UPHS-029ZE QuantiNova LNA | QIAGEN | 249955 |
| Probe Focus Panels | | |

### 3. Instrumentation of In-Vitro Analysis

| Instrument | Vendor | Model |
|---|---|---|
| Countess | Invitrogen | |
| Centrifuge | Eppendorf | 5810R |
| Vortex | IKA | MS3 digital |
| QuantStudio 7 Flex Real- | Applied Biosystems | 4484643 |
| Time PCR System | | |

### 4. Cell Line

| Cell Line | Culture Medium |
|---|---|
| OVCAR3 | RPMI-1640 + 0.01 mg/ml |
| | insulin + 20% FBS |

Cell Seeding: OVCAR3 cells from flask were harvested into cell culture medium and then cell number was counted. Cells were diluted with culture medium for 300000 cells/well and 2000 μL of cell suspension was added into each well of 6-well cell culture plate. Plates were covered with lid and placed in room temperature for 30 minutes after gently shaking and then transferred into 37° C. 5% CO₂ incubator overnight for cell attachment.
Compound Treatment: E7820 was dissolved at 10 mM DMSO stock solution and diluted to 3 mM by transferring 6 μL of stock solution into 14 μL DMSO. Compound was then diluted to 0.3 mM by transferring 2 μL of 3 mM diluted compound into 18 μL DMSO. 2 μL of diluted compound was transferred from compound source plate into the cell plate with the final concentration was 0.3 μM and 3 μM, respectively. For the negative control, 2 μL DMSO was used. Plates were shaken gently at room temperature at 200 RPM for 1 minute. Compounds treatment for 24 hours in 37° C. 5% CO₂ incubator.
Cell Harvest & Storage: Lysis buffer volume with 1% β-ME (600 μl/well of 6-well plate) was prepared. After 24 hrs of compound treatment, media was removed by aspiration and cells washed in each well by 2 ml of cold 1×PBS. 600 μl of lysis buffer (+1% β-ME) was added to each well of 6-well plate. A 1000 μl pipettor was used to transfer the cell lysate (˜600 μl) to the labeled 1.5 ml vials. Next, 70% Ethanol with Nuclease free water was prepared. 1 volume (600 μl) of 70% ethanol was added to each vial and mixed well by pipetting up and down 3 times. Lysates were loaded onto QiaShredder column and spun at 12000 rpm for 15 seconds. Flow-through collected and remaining 600 μL sample was transferred to same column, Flow-through collected. 600 μL of the flow-through sample was transferred to an RNeasy spin column placed in a 2 mL collection tube, Centrifuged at 12000 rpm at RT for 15 s. and flow-through discarded. Remaining 600 μL sample transferred to same column, Centrifuged at 12000 rpm at RT for 15 s. and flow-through discarded. RNeasy spin column placed in a new collection tube. 500 μL of wash buffer added to the RNeasy spin column and centrifuged at 12000 rpm at RT for 15 s, with flow-through discarded. Centrifuged again at 12000 rpm at RT for 2 min. RNeasy spin column placed in a new collection tube with lid. 30 μL of Rnase-free water was added directly to the membrane of the column and Incubated at RT for 2 min, followed by centrifuge at 12000 rpm at RT for 2 min. RNA samples spec using Nanodrop and RNA was stored at −80° C.
RNA Extraction and qPCR Performance: RNA total and RNA samples were thawed on ice. RT Master Mix (10 μl 2×RT buffer+1 μl 20×RT enzyme mix) was prepared at RT. 11 μL of RT Master Mix was distributed to each well of 96-well qPCR plate y pipette by row. 2 μg of the RNA samples were transferred to the 96-well qPCR plate by pipette. In addition, 2 μg of the RNA samples and 2 μL internal control RNA were transferred to the 96-well qPCR plate by pipette. Total volume is 20 per reaction, so the remaining volume was filled with H20. Plate was spun for 1 minute and RT reaction was performed (Incubate at 37° C. for 1 hour then at 95° C. for 5 minutes to deactivate the enzyme. Hold indefinitely at 4° C.) qPCR cocktail was prepared at RT.

| | Volume for 1 |
|---|---|
| Reagents | reaction (uL) |
| TaqMan Fast Advanced | 5 |
| Master Mix (2X) | |
| Nuclease-free water | 4.5 |
| cDNA | 0.5(50 ng) |
| Total volume per reaction | 10 |

10 μL qPCR cocktail was distributed in to the corresponding well of a UPHS-029ZE QuantiNova LNA Probe Focus Panels (QIAGEN, 249955). Reaction was sealed with optical adhesive film, then centrifuged briefly to bring the PCR reaction mix to the bottom of the plate. Experiment was then set up using the following conditions.

| UDG | Enzyme | | |
|---|---|---|---|
| activation | activation (hold) | PCR (40 cycles) | |
| Hold | Hold | Denature | Anneal/extend |

| | | | | | | | |
|---|---|---|---|---|---|---|---|
| 50° | C. | 95° | C. | 95° | C. | 60° | C. |
| 2 | min | 20 | s | 1 | sec | 20 | sec |

Calculation: Normalized fold expression. Normalized fold expression calculation is known in the prior art. ΔCT was calculated by using following equation: ΔCT=CT_{Target}−CT_{Reference genes}. Relative mRNA expression (ΔΔCT) was calculated as ΔΔCT=2^{-ΔCT}. Fold expression was then normalized to the geometric mean of 5 housekeeping genes (ACTB, B2M, GAPDH, HPRT1, and RPLPO) with fold expression calculated (Taylor S C, et al. Trends Biotechnol., 2019, 37(7): 761-774)

### Results

Gene expression levels for BRCA1 and ATR following 6-hour treatment with E7820 in OVCAR3 cells are shown across 5 doses in FIG. 10A. Gene expression levels for a network of DNA repair genes (RAD50, RAD18, TOPBP1, FANCD2, XRCC1), cell checkpoint genes (ATM, CHEK1) and CDK7 following 24-hour treatment with E7820 at 0.03 μM in OVCAR3 cells are shown in FIG. 10B. RBM39 degradation shows dose-dependent decreases in critical genes regulating homologous recombination and cell cycle checkpoints; however, targets such as CHEK1 and CDK7 remain relatively stable suggesting RBM39 degradation is capable of inducing specific downregulation of DDR genes while not impacting expression of genes attributed to dose limiting toxicities such CHEK1 and CDK7. These figures demonstrate that RBM39 degradation is sufficient to induce a BRCAness phenotype in-vitro.

### Example 8—Efficacy and Target Engagement in Balb/C Nude Mice Treated with Compound A Alone and in Combination with PARP Inhibitor Niraparib in the BACA-Proficient Ovarian Cancer OV0273 PDX Model

### Materials and Methods

Tumor Inoculation: Tumor fragments from female Balb/C nude mice were harvested and used for inoculation into mice. Each mouse was inoculated subcutaneously in the right upper flank with primary human ovarian tumor xenograft model OV0273 tumor fragment (2-3 mm in diameter) for tumor development.
Randomization: The randomization starts from when the mean tumor size reaches approximately 150˜200 mm³. 32 mice are enrolled in the study for the in vivo efficacy portion and 12 mice are enrolled in the study for the pharmacodynamic tumor marker portion. All animals are randomly allocated to 4 study groups. Randomization is performed based on “Matched distribution” method. The date of randomization is denoted as day 0. Tumor sizes after randomization across different treatment groups is shared.
Western Blotting: Methods of western blot are known in the art. Tumor tissue samples from mice were transferred into 5 ml polypropylene tubes, weighed, and prepared with 3× volume lysis buffer (e.g., 100 mg tumor sample with 300 ul lysis buffer). Samples were homogenized and then lysed for 30 min. Samples were then centrifuged at 14,000 g for 15 min at 4° C. Supernatant was transferred to a fresh tube. Protein quantification was performed according to Pierce BCA Protein Assay Kit (ThermoFisher). 50 ug/lane of each PDX tumor protein lysate was loaded into pre-cast gels (26 wells, 4-15%, Bio-Rad Criterion TGX). Gels were run with constant voltage (60V). PVDF membrane was pre-activated in methanol for 2 min, followed by pre-wetting membranes and filter paper in cold transfer buffer. Gel membrane was sandwiched, and protein transfer was started for 2 hr at 280 mA. Primary antibody was diluted (1:1000) in TBST with 5% milk and incubated overnight at 4° C. with gentle shaking. Membranes were washed in TBST 3 times for 5 minutes each. Membranes were then incubated with secondary antibody diluted in TBST with 5% milk for 1 hr at RT on a nutator. Membranes were washed in TBST 3 times for 5 minutes and target proteins were detected with Tanon 5200 chemiluminescence image analysis system using the ECL method. Primary antibody used to detect RBM39 was obtained from MEC (Cat #HPA001591). Caco-2 treated cells were used as a positive control.
Tumor Growth Inhibition (TGI): Methods of assessing TGI are known in the art. Tumor volumes were measured three times per week after randomization in two dimensions using a caliper, and the volume was expressed in mm³ using the formula: V=(L×W×W)/2, where Vis tumor volume, L is tumor length (the longest tumor dimension) and W is tumor width (the longest tumor dimension perpendicular to L). % TGI was calculated using the formula % TGI=(TV vehicle−TV treatment)/(TV vehicle−TV initial)*100 for all mice (Wong, H, et al. Clin Cancer Res., 2012, 18(14): 3846-3855).
Treatment Arms: 8 mice per arm (efficacy) or 3 mice per arm (PD marker) were treated with either Vehicle, niraparib, Compound A, or Compound A plus niraparib, for 28 days (efficacy) or 3 days (PD marker). Niraparib was administered PO QD at 40 mg/kg and Compound A was administered PO BID at 100 mg/kg. Vehicle formulation matched Compound A. BID timepoint for Compound A was 12 hours.

### Results

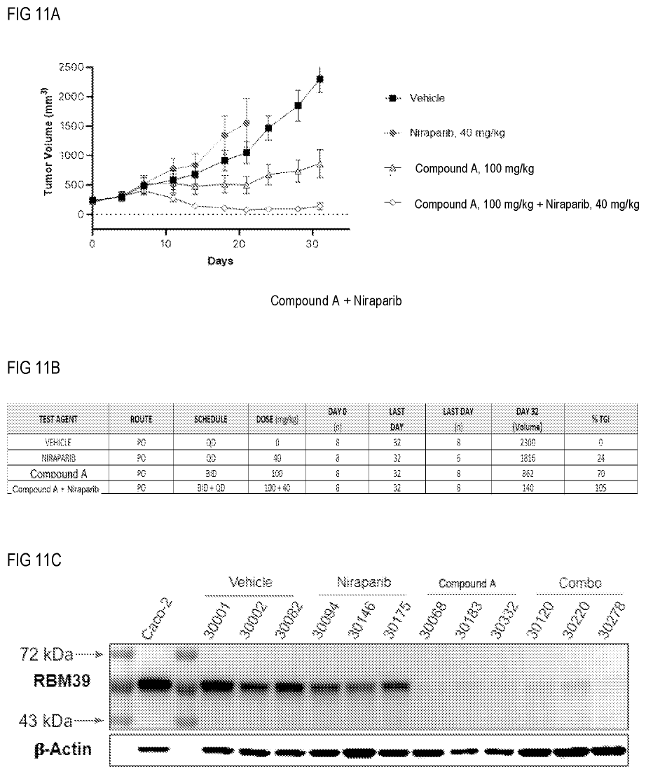

The results are shown in FIG. 11A (tumor volume) and FIG. 11B (Tumor Growth Inhibition (TGI) and FIG. 11C (western blot). FIG. 11A shows that tumor volume was significantly decreased compared with vehicle with either single agent Compound A (p<0.001) or the combination of Compound A plus niraparib (p<0.001) in the BRCA-proficient OV0273 PDX model. This model was resistant to treatment with niraparib, while Compound A alone was superior to niraparib alone (p<0.05). RBM39 degradation alone or in combination with PARP inhibition was therefore superior to treatment with PARP inhibition alone in the BRCA-proficient ovarian PDX mouse model. Statistical analysis between groups was conducted using one-way ANOVA with Tukey's multiple comparison's test. Data was analyzed in GraphPad Prism 10.1.1. FIG. 11C shows that Compound A is capable of potently degrading RBM39 levels in the tumors of mice treated after 3 days.

1. A method for treating (i) a homologous recombination (HR)-deficient cancer, (ii) a homologous recombination (HR)-proficient cancer or (iii) a cancer that is resistant to DNA repair and DNA damage response (DDR) inhibitor therapy, in a subject in need thereof, the method comprising administering to the subject an RNA-binding motif protein 39 (RBM39) degrader in an amount effective to treat the cancer.

2. The method according to claim 1, further comprising administering a DNA repair and DNA damage response (DDR) inhibitor to the subject in an amount effective to treat the cancer.

3. The method according to claim 2, wherein the DDR inhibitor is a Poly (ADP-Ribose) Polymerase (PARP) inhibitor.

4. The method according to claim 3, wherein the PARP inhibitor is olaparib.

5. The method according to claim 1, wherein the cancer that is resistant to DDR inhibitor therapy is resistant to Poly (ADP-Ribose) Polymerase (PARP) inhibitor therapy.

6. The method according to claim 1, wherein the cancer is selected from the group consisting of ovarian cancer, breast cancer, prostate cancer, gastric cancer, pancreatic cancer, a KRAS mutated cancer, acute myeloid leukemia, colon cancer, neuroblastoma, hematopoietic cancers, lymphoid cancers, non-small cell lung cancer and small-cell lung cancer.

7. The method according to claim 6, wherein the cancer is ovarian cancer.

8. The method according to claim 1, further comprising determining the homologous recombination deficiency (HRD) status of the subject.

9. The method according to claim 1, further comprising determining the presence or absence of one or more deleterious mutations in one or more genes selected from the group consisting of breast cancer susceptibility genes 1 or 2 (BRCA1 or BRCA2), genes related to the Fanconi Anemia repair pathway, ATM, TP53, genes related to the base excision repair pathway, genes related to the non-homologous end joining pathway and genes related to the alternative-end joining pathway.

10. The method according to claim 1, further comprising determining the presence or absence of one or more deleterious mutations in BRCA1 and/or BRCA2.

11. The method according to claim 1, further comprising assessing the subject for overexpression or amplification of RBM39 and/or DDB1 and CUL4 associated factor 15 (DCAF15).

12. The method according to claim 1, wherein the RBM39 degrader is an aryl sulfonamide.

13. The method according to claim 12, wherein the aryl sulfonamide is E7820.

14. The method according to claim 12, wherein the aryl sulfonamide is Compound A.

15. A composition comprising an RNA-binding motif protein 39 (RBM39) degrader and a DNA repair and DNA damage response (DDR) inhibitor.

16. The composition according to claim 15 wherein the DDR inhibitor is a poly (ADP-ribose) polymerase (PARP) inhibitor.

17. The composition according to claim 15, wherein the RBM39 degrader is an aryl sulfonamide.

18. The composition according to claim 17, wherein the aryl sulfonamide is E7820.

19. The composition according to claim 17, wherein the aryl sulfonamide is Compound A.

20. A method of predicting a subject's response to cancer treatment with an RNA-binding motif protein 39 (RBM39) degrader, the method comprising determining the subject's response to treatment with a DNA repair and DNA damage response (DDR) inhibitor therapy and, if the subject is identified as being resistant to DDR inhibitor therapy, administering an RBM39 degrader to the subject in an amount effective to treat the cancer.

21. The method according to claim 20, further comprising administering a DNA repair and DNA damage response (DDR) inhibitor to the subject in an amount effective to treat the cancer.
